(19) United States
(12) Patent Application Publication
(10) Pub. No.: US 2014/0081109 A1
(43) Pub. Date: Mar. 20, 2014

(54) Microdialysis Sampling/Delivery Device

(71) Applicant: Mehul J. DESAI, Silver Spring, MD (US)

(72) Inventor: Mehul J. DESAI, Silver Spring, MD (US)

(21) Appl. No.: 14/027,774

(22) Filed: Sep. 16, 2013

Related U.S. Application Data
(60) Provisional application No. 61/701,203, filed on Sep. 14, 2012.

(51) Int. Cl. A61B 5/145; A61B 5/157; A61B 5/00; A61B 5/155

(52) U.S. Cl. CPC A61B 5/14528; A61B 5/155; A61B 5/14546; A61B 5/157; A61B 5/4566
USPC 600/366; 600/581

(57) ABSTRACT

This present invention provides real-time in vivo sampling via ultra-small volume microdialysis of the intervertebral disc to assay single molecules of interest. The invention consists of three lumena, two that are capped with a membrane capable of sampling tissues via diffusion. A guide wire can provided between these two lumena so that they may be extended beyond the housing of the three lumena and directed via the guide wire. The third lumen can be utilized for injection or aspiration. Theoretically, agents including treatments such as stem cells or pharmaceutical agents may be introduced to the disc via this third lumen and the real-time effects may be assayed with the first two lumena. The three lumena may be placed adjacent to each other or in a concentric fashion to minimize the total size of the device.

### RELATED APPLICATIONS

This application claims the benefit of U.S. Provisional Application No. 61/701,203, filed Sep. 14, 2012, the entire contents of which are incorporated herein by reference.

### BACKGROUND OF THE INVENTION

### Internal Disc Degeneration

Low back pain (LBP) is an extremely common problem affecting approximately 80-90% of the U.S. population at some point in their lives (1). An estimated indirect annual cost of $16-50 billion makes it the costliest musculoskeletal problem in the U.S. (2). Additionally chronic LBP is the leading cause of disability in individuals under age 45 and the third leading cause in those over 45 (3). Although LBP tends to resolve spontaneously, 70-90% of patients with a previous episode of LBP will experience a recurrence.

Among various etiologies, discogenic pain mediated by internal disc disruption (IDD) is the most common cause of chronic low back pain; it has been implicated in up to 40% of patients with LBP (4). IDD was first described by Crock in 1970 as a condition marked by alteration in the internal structure and metabolic functions of the intervertebral disc (IVD), usually preceded by injuries to the annulus fibrosis with resultant annular tears including radial and circumferential tears which are the major forms of IDD. (5)

The role of cytokines as mediators in disc degeneration has been somewhat elucidated. Investigators have found that IVD cells have the capability of producing an array of cytokines including but not limited to interlukin(IL)-1β, IL-6, IL-8 and tumor necrosis factor alpha (TNFα). IL-1β has been found in the NP of human IVD (18), in addition to herniated, degenerative and displaced discs. IL-1β is hence implicated as a component in IVD degeneration. Increases in proteases gene expression, with affinities to Type II collagen and aggrecan, are exhibited in the presence of IL-1β. Type II collagen and aggrecan are two important components for integrity and are found in high concentrations in the NP. Additionally, other interleukins (IL) have been investigated, including IL-6 and IL-8. IL-6 functions in proteoglycan synthesis inhibition in cartilage while IL-8 is known to function in angiogenesis and chemically attract and activate neutrophils. Burke et al examined extracted discs and the production of pro-inflammatory mediators. Elevated levels of both of IL-6 and IL-8 were recorded from surgically extracted discs (19). IL-8 production was elevated in extruded and sequestered human discs compared to control and annulus intact herniations (20). Similarly, TNFα was also found to be present in cells of both the NP and AF (17, 21). Symptomatic human discs studied demonstrated a greater number of TNFα producing cells compared to controls (21). Implicated in disc herniation and sciatic pain, studies have suggested TNFα as a player in herniated nucleus pulposus induced nerve root damage and pain (17, 22). In painful discs, substance P has been found in both the margins of annulus fibrosus tears and within granulation tissue in the nucleus pulposus (26). One study demonstrated immunoreactivity to calcitonin gene-related peptide and substance P in annuli fibrosi, but not in the nucleus pulposus (27).

The major limitations of these previous investigations are related to acquisition of data through cadaveric and surgical specimens. To date real-time in-vivo sampling and quantification has not been available, furthermore investigation at the level of single molecules at low volumes has not been available.

The role of various cytokines and neuropeptides within the degenerative cascade of the intervertebral disc remains poorly elucidated. Increasingly additional molecules are identified via cadaveric and surgical examination but their involvement in the initiation and potentiation of degeneration remains only partially described. Furthermore technologically advanced treatment options are currently based and developed on this rudimentary understanding of the intervertebral disc.

### SUMMARY OF THE INVENTION

This present invention provides real-time in vivo sampling via ultra-small volume microdialysis of the intervertebral disc to assay single molecules of interest. The invention consists of three lumena, two that are capped with a membrane capable of sampling tissues via diffusion. A guide wire can be provided between these two lumena so that they may be extended beyond the housing of the three lumena and directed via the guide wire. The third lumen can be utilized for injection or aspiration. Theoretically, agents including treatments such as stem cells or pharmaceutical agents may be introduced to the disc via this third lumen and the real-time effects may be assayed with the first two lumena. The three lumena may be placed adjacent to each other or in a concentric fashion to minimize the total size of the device.

### BRIEF DESCRIPTION OF THE FIGURE

FIG. 1 is shows an axial section through a device exhibiting the key components in accordance with a preferred embodiment of the invention; and

FIG. 2 is a cross-sectional view of the device.

### DETAILED DESCRIPTION OF THE PREFERRED EMBODIMENTS

In describing the preferred embodiments of the present invention illustrated in the drawing, specific terminology is resorted to for the sake of clarity. However, the present invention is not intended to be limited to the specific terms so selected, and it is to be understood that each specific term includes all technical equivalents that operate in a similar manner to accomplish a similar purpose.

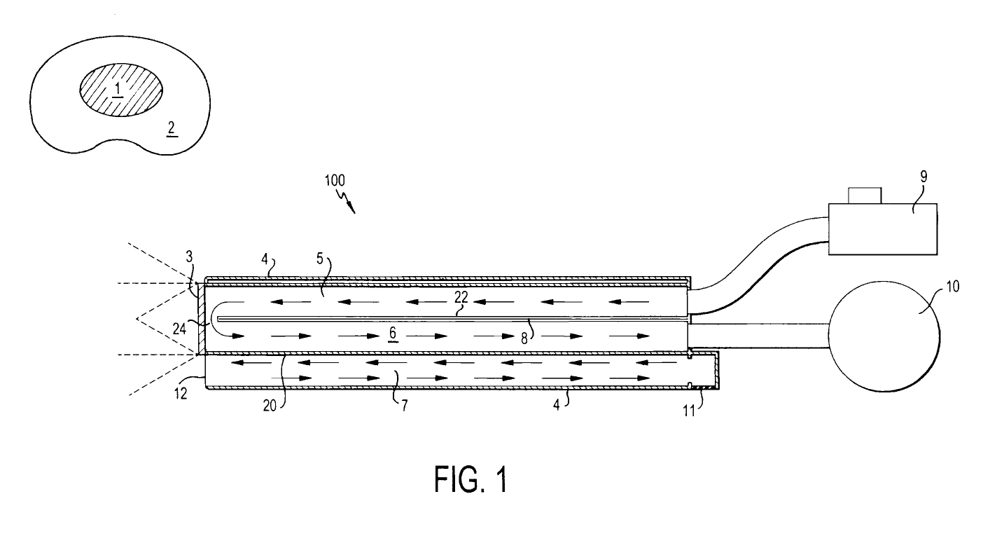

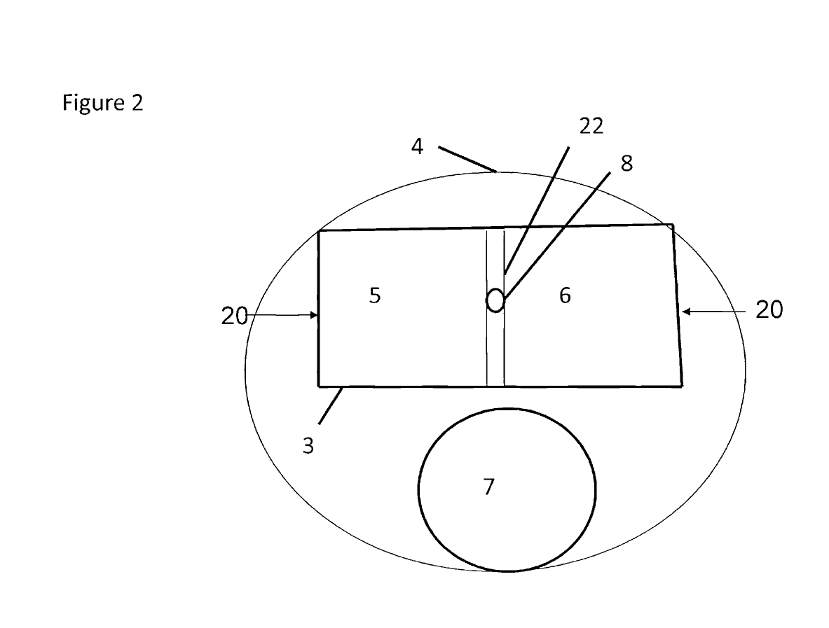

FIGS. 1 and 2 show a microdialysis sampling and delivery device 100 in accordance with an embodiment of the invention. The device 100 is intended for insertion into biological tissues, such as intradiscal placement which comprises a series of lumina and membranes. The device generally includes a membrane 3, outer wall 4, inner wall 20, inflow lumen 5. outflow lumen 6, injection/aspiration lumen 7, guide wire 8, injection port 11 and opening 12.

The device has an external fixed shell, i.e. outer wall 4. The outer wall 4 can be rigid, or semi-rigid and is constructed of metal, plastics or other materials to provide protection for the analytical device. The outer wall 4 can have a circular cross-section, so that the device 100 is generally an elongated tubular shape. It will be appreciated that any suitable material can be utilized having different strength, rigidity and pliability.

The outer wall 4 defines an enclosure having an inner space that contains at least three lumina: an inflow lumen 5, an outflow lumen 6, and an injection/aspiration lumen 7. The inflow and outflow lumen 5, 6 are contained within the inner wall 20 (which is contained within the outer wall 4) or may be separate (as shown), and at least a portion of the inner wall 20 can be directly in contact with a portion of the outer wall 4. The inner wall 20 can have a similar cross-sectional shape as the outer wall 4 such as a circle to form a circular tube. Or, the inner wall 20 can have a different cross-sectional shape such as a rectangle with a top inner wall, bottom inner wall and two opposing inner side walls (as shown in FIG. 2).

An internal dividing wall 22 is provided between the inflow and outflow lumina 5, 6, within the inner wall 20. The internal dividing wall 22 optionally contains a guide wire 8. The two lumina 5, 6 are thereby configured to carry a dialysate mixture. Preferably, those inflow and outflow lumina 5, 6 are adjacent one another, as shown. The lumina 5, 6 are separated from each other by the internal wall 22 and/or the guide wire 8 or other barrier so that fluid cannot move between the lumina 5, 6 except around the distal end of the guide wire 8. That is, the dividing wall 22 is impermeable so that the liquid or material in the lumina 5, 6 cannot pass through the dividing wall 22. The dividing wall 22 is shown in FIG. 2 as being two separate walls that extend vertically within the inner walls. However, the dividing wall can be a single wall that is either a thicker wall to allow for the guide wire 8 or a thinner wall with the guide wire 8 located elsewhere within the device 100. In addition, the dividing wall(s) 22 can be positioned horizontally or at another position within the inner walls 20. The dividing wall(s) 22 are further show being linear and separating the interior space within the inner walls 20 in half so that the inflow lumen 5 is about the same size as the outflow lumen 6. However, the dividing wall 22 can be configured to be non-linear and/or to separate the interior space so that one of the lumen 5, 6 is larger than the other. Still yet, the inflow lumen 5 and outflow lumen 6 need not be immediately adjacent to and touching one another (and contained within or sharing at least a portion of the inner wall(s) 22), but can be entirely separate, each with one or more respective inner walls 22.

The inner wall 20 essentially forms an inner elongated container or tube that is located within the outer elongated container or tube defined by the outer wall 4. The inner tube can move independent of the outer tube, so that, for instance, the inner tube can extend outward beyond the outer tube and be placed more precisely with respect to the target tissue 1, 2. Thus, the inner tube can slide inward and outward with respect to the outer tube. In addition, the inner tube can be rotated within the outer tube or once deployed beyond the inner tube.

The internal dividing wall 22 has a distal end that terminates just proximal to a microdialysis membrane 3. In this manner and as shown by the arrows in FIG. 1, the fluid, gas other material (preferably a dialysate mixture) may flow in through the inflow lumen 5, around the distal end of the guide wire 8 between the distal end of the guide wire 6 and the membrane 3, and back in through the outflow lumen 6. The cellulose ester microdialysis membrane 3 is semi-permeable and allows the transfer of substances of appropriate sizes (˜100,000 kDaltons). The membrane 3 is fixed at the terminal ends of the inflow and outflow lumina 5, 6. The membrane 3 is affixed over the distal ends of the two lumina 5, 6 at a distance of approximately 200 μm between the distal end of the internal dividing wall 22 and the membrane 3. This creates a space 24 between the guide wire 8 and the membrane 3 through which material can pass from the inflow lumen 5 to the outflow lumen 6. The membrane 3 is attached to a distal end surface of the inner wall(s) 20 of the middle lumina 5, 6. The membrane 3 can be cemented or glued in place to the inner wall(s) 20.

Furthermore, an injection/aspiration lumen 7 is positioned adjacent to the inflow and outflow lumina 5, 6, and terminates at the end of the outer wall 4. Variations of this design include a device with the sampling unit retracted within the outer wall 4 or one with the sampling unit beyond the outer wall. Furthermore, the sampling unit may be deployable and steerable (via the guide wire) to analyze structure or tissues adjacent to the fixed shell. Typically the external fixed shell 4 is placed via a delivery device such as a needle. The external shell 4 and the accompanying lumina 5, 6, 7 can be of variable lengths likely 5-8 inches in length. The shell will likely be 25-27 gauge but may be variable. As illustrated in FIG. 2, the aspiration lumen 7 can be separate from the inflow and outflow lumen 5, 6, or can touch the inflow and outflow lumen yet would still consist of its own walls that would not be permeable to the other two lumena 5, 6.

This device 100 can be delivered to target tissues 1, 2 through a needle to protect its integrity. While the invention has been shown and described as having three lumina 5, 6, 7, more lumen can be provided. Preferably however, at least one lumen (here shown as the injector/aspiration lumen 7) of a minimum of the three total lumen 5, 6, 7 is dedicated to delivery of substances into the target tissues 1, 2 or aspiration of substances from the target tissue 1, 2. And, the injection lumen 7 can be provided within its own tube that includes another inner wall container. The injection tube can be positioned inside the outer wall 4 of the housing and outside of the inner tube of the inflow/outflow lumina 5, 6, as shown.

In addition, while the guide wire 8 is shown between the two lumina 5, 6, it can be located along the outer wall 4, or between the lumen 5, 6 and the injection/aspiration lumen 7. Other suitable variations are also within the scope of the invention. For instance, though the inflow and outflow lumina 5, 6 are shown in a side-by-side relationship, they can be in a concentric relationship, such as with the inflow lumen 5 at the center surrounded by the outflow lumen 6, and with the injector lumen 7 to the side or concentric thereto. And, the inner wall 20 can have a circular or oval cross section.

Furthermore, the distal element of the microdialysis sampling unit 10 can either project beyond the outer wall 4 (as shown), or lay within the outer wall 4. Also the distal element may be housed with the supporting structure at placement but may be deployable and steerable in a circular fashion to a set distance via a steering mechanism such as a guide-wire 8 built into the wall 22 separating the dialysis lumina 5, 6. For instance, the wall 22 between the two sampling lumina can be reinforced and house a guide-wire mechanism 8 in a hollow structure. A dialysis pump 9 is provided to drive a solute through the inflow lumen 5 to sample and/or exchange substances from the target tissue at the site of the membrane. The solute will return through the outflow lumen 6 to the collection chamber 10. Thus, the pump 9 is in fluid communication with the inflow lumen 5, and the collection unit 10 is in fluid communication with the outflow lumen 6. The collection unit 10 can be, for instance, a well or a series of wells that collects the sample.

In operation, the device 100 allows for the simultaneous sampling of biological substances. Furthermore this may be done prior to and after injection in a real-time fashion. A dialysate mixture or solute is introduced into the inflow lumen 5 by the pump 9. The solute flows to the distal end of the inflow lumen 5, where the pH difference between the solute and the target tissue 1, 2 across the membrane derives the diffusion. At the same time, a medication or biologic agent (or other substance to be studied) may be introduced to the target tissue 1, 2 through the injector lumen 7. The tissue 1, 2 reacts to the medication or biologic agent, and the biological substances that would react to that injectate would be sampled across the membrane 3. The membrane 3 works by simple diffusion, whereby a gradient across the membrane (i.e., pH of solute different than pH of intradiscal space) allows the diffusion of biological substance across it to be sampled. This gives relative levels of these biological substances within the disc via real-time in vivo sampling. The membrane 3 does not allow the dialysate or solution to cross into the IVD (so that the dialysate or solution stays in the lumena 5, 6, and only the gradient allows the molecules of interest to cross over. Biological substances that are problematic might also be removed. That mixture is then collected through the outflow lumen 6, and deposited in the collection unit 10.

Thus, the device 100 allows substances beyond the membrane 3 to be sampled. The dialysis pump 9 moves the fluid at a predetermined rate sufficient to move the fluid from the inflow lumena 5 past the membrane 3 into the outflow lumena 6 and into the collection reservoir 10. However, the rate is sufficiently slow to allow appropriate diffusion. The membrane 3 is only over lumina 5, 6 because that is the entirety of the sampling unit. The membrane 3 is not placed over the injector lumen 7 because it is needed for injection or possibly aspiration. A syringe is attached to the injection port 11 possibly via a connector tube to inject or aspirate samples.

It should be noted that although the arrows in FIG. 1 show movement both in and out of the injection/aspiration lumen 7, only one movement occurs at a given time. That is, a substance can be injected into the lumen 7, which moves toward the target tissue 1, 2 (right to left in the embodiment shown). Or, substance can be aspirated out of the lumen, which moves away from the target tissue 1, 2 (left to right in the embodiment of FIG. 1).

The pump 9 preferably provides a slow flow rate of about 1 microliter to 1 milliliter per minute. The desired flow rate can vary depending on the sensitivity of the assay. The invention is able to perform micro-dialysis with a very small volume of sample, under 1 milliliter and as little as 1 microliter. Of course, any suitable flow rate and sampling volume can be provided.

The present invention is able to elucidate the role of various cytokines and neuropeptides within the degenerative cascade of the intervertebral disc. The device 100 can obtain samples at individual molecule using the membrane 3. The invention is able to obtain samples of the target tissue 1, 2 in vivo and determine, for instance, how the IVD changes when a biological agent injected. Thus, in vivo changes in the IVD neurotransmitters can be analyzed before and after a biological agent is administered to the target tissue 1, 2.

The device 100 samples tissue locations such as the intradiscal milieu in a real-time fashion. Biological tissues contain various cytokines, neuropeptides and substances engaged in the transport of signals in a catabolic or anabolic state. In order to understand the functioning in structures such as the intervertebral disc, which as this time we only understand from cadaveric and surgical specimens, elucidating the real-time relative values of biological substances within these tissues may provide insight. The device 100 is steerable and deployable.

The following documents are incorporated herein by reference:

1. Quebec Task Force on Spinal Disorders: A Monography for Physicians. Spine 1987; 12:551-9.
2. Spengler D M, Bigos S J, Martin N A, et al: Back Injuries in Industry: A Retrospective Study. I: Overview and Cost Analysis. Spine 1986; 11:241-5.
3. Andersson G B: Epidemiological Features of Chronic Low Back Pain. Lancet 1999; 354:581-5.
4. Crock H V. Internal Disc Disruption. A Challenge to Disc Prolapse Fifty Years On. Spine 1986; 11:650-3.
5. Schwarzer A C, Aprill C N, et al. The Prevalence and Clinical Features of Internal Disc Disruption in Patients with Chronic Low Back Pain. Spine 1995; 17:1878-83.
6. Bogduk N. Clinical Anatomy of The Lumbar Spine and Sacrum. 3^{rd} Ed. Edinburgh, Scotland: Churchill Livingstone; 1997:205-212.
7. Cohen S P, Larkin T M, Barna S A, et al. Lumbar Discography: A Comprehensive Review of Outcome Studies, Diagnostic Accuracy, and Principles. Reg Anes Pain Med 2005; 2(30):163-183.
8. Lotz J C. Chin J R. Intervertebral disc cell death is dependent on the magnitude and duration of spinal loading. Spine 2000 Jun. 15; 25(12):1477-83.
9. Setton L A, Chen J. Mechanobiology of the intervertebral disc and relevance to disc degeneration. J Bone Joint Surg Am. 2006 April;88 Suppl 2:52-7.
10. Iatridis J C. Mente P L. Stokes I A. Aronsson D D. Alini M. Compression-induced changes in intervertebral disc properties in a rat tail model. Spine 1999 May 15:24(10):996-1002.
11. MacLean J J. Lee C R. Alini M. Iatridis J C. The effects of short-term load duration on anabolic and catabolic gene expression in the rat tail intervertebral disc. Journal of Orthopaedic Research. 23(5):1120-7, 2005 September.
12. Boos N. Weissbach S. Rohrbach H. Weiler C. Spratt K F. Nerlich A G. Classification of age-related changes in lumbar intervertebral discs: 2002 Volvo Award in basic science. Spine. 27(23):2631-44, 2002 Dec. 1.
13. Cs-Szabo G. Ragasa-San Juan D. Turumella V. Masuda K. Thonar E J. An H S. Changes in mRNA and protein levels of proteoglycans of the anulus fibrosus and nucleus pulposus during intervertebral disc degeneration. Spine. 27(20):2212-9, 2002 Oct. 15.
14. Weiler C, Nerlich A G, Zipperer J, Bachmeier B E, Boos N. 2002 SSE Award competition in Basic Science: expression of major matrix metalloproteinases is associated with intervertebral disc degradation and resorption. Eur Spine J. 2002 August;11(4):308-20.
15. Kang J D. Stefanovic-Racic M. McIntyre L A. Georgescu H I. Evans C H. Toward a biochemical understanding of human intervertebral disc degeneration and herniation. Contributions of nitric oxide, interleukins, prostaglandin E2, and matrix metalloproteinases. Spine. 22(10):1065-73, 1997 May 15.
16. Olmarker K, Rydevik B, Nordborg C. Autologous nucleus pulposus induces neurophysiologic and histologic changes in porcine cauda equina nerve roots Spine. 1993 Sep. 1; 18(11):1425-32.
17. Olmarker K, Larsson K. Tumor necrosis factor alpha and nucleus-pulposus-induced nerve root injury. Spine. 1998 Dec. 1; 23(23):2538-44.
18. Le Maitre C L, Freemont A J, Hoyland J A. The role of interleukin-1 in the pathogenesis of human intervertebral disc degeneration. Arthritis Res Ther. 2005 Apr. 1; 7(4):R732-45.
19. Burke J G, Watson R W, McCormack D, Dowling F E, Walsh M G, Fitzpatrick J M. Intervertebral discs which cause low back pain secrete high levels of proinflammatory mediators. J Bone Joint Surg Br. 2002 March;84(2):196-201.
20. Burke J G, Watson R W, McCormack D, Dowling F E, Walsh M G, Fitzpatrick J M. Spontaneous production of monocyte chemoattractant protein-1 and interleukin-8 by the human lumbar intervertebral disc. Spine. 2002 Jul. 1; 27(13):1402-7.
21. Weiler C, Nerlich A G, Bachmeier B E, Boos N. Expression and distribution of tumor necrosis factor alpha in human lumbar intervertebral discs: a study in surgical specimen and autopsy controls. Spine. 2005 Jan. 1; 30(1):44-54.
22. Igarashi, Tamaki, Kikuchi, Shinichi, Shubayev, Veronica, Myers, Robert R. Exogenous Tumor Necrosis Factor-Alpha Mimics Nucleus Pulposus-Induced Neuropathology: Molecular, Histologic, and Behavioral Comparisons in Rats. Spine 2000 Dec. 1; 25(23):2975-2980.
23. Holmsen H, Weiss H J. Secretable storage pools in platelets. Annu Rev Med. 1979;30:119-34.
24. Abbott F V, Hong Y, Blier P. Activation of 5-HT_{2A} receptors potentiates pain produced by inflammatory mediators. Neuropharmacology, 1996 January:35(1):99-110.
25. Kanayama M, Hashimoto T, Shigenobu K, Oha F, Yamane S. New treatment of lumbar disc herniation involving 5-hydroxytryptamine2A receptor inhibitor: a randomized controlled trial. J Neurosurg Spine. 2005 April;2(4):441-6.
26. Peng B, Wu W, Hou S, Li P, Zhang C, Yang Y. The pathogenesis of discogenic low back pain. J Bone Joint Surg Br. 2005 January;87(1):62-7.
27. Ashton I K. Roberts S. Jaffray D C. Polak J M. Eisenstein S M. Neuropeptides in the human intervertebral disc. Journal of Orthopaedic Research. Mar. 1994 2(2):186-92.
28. Lindblom K. Diagnostic puncture of intervertebral disks in sciatica. Acta Orthop Scand 1948:17:231-239.
29. Merskey H, Bogduk N. Classification of Chronic Pain: Descriptions of Chronic Pain Syndromes and Definitions of Pain Terms. Seattle, Wash.: IASP Press; 1994:180-181.
A. Doi-Saika M, Tokunaga A, Senba E. Intradermal 5-HT induces Fos expression in rat dorsal horn neurons not via 5-HT3 but via 5-HT2A receptors. Neurosci Res. 1997 October;29(2):143-9.
B. Giordano J. Dyche J. Differential analgesic actions of serotonin 5-HT3 receptor antagonists in the mouse. Neuropharmacology. 1989 April; 28(4):423-7.
C. Giordano J. Rogers L V. Peripherally administered serotonin 5-HT3 receptor antagonists reduce inflammatory pain in rats. European Journal of Pharmacology. 1989 Oct. 24; 170(1-2):83-6.
D. Karppinen J, Korhonen T, Malmivaara A, Paimela L, Kyllonen E, Lindgren K A, Rantanen P, Tervonen O, Niinimaki J, Seitsalo S, Hurri H. Tumor necrosis factor-alpha monoclonal antibody, infliximab, used to manage severe sciatica. Spine. 2003 Apr. 15; 28(8):750-4.
E. Sufka K J. Schomburg F M. Giordano J. Receptor mediation of 5-HT-induced inflammation and nociception in rats. Pharmacology, Biochemistry & Behavior. 1992 January;41(1):53-6.
F. Taiwo Y O, Levine J D. Serotonin is a directly-acting hyperalgesic agent in the rat. Neuroscience. 1992;48(2):485-90.
G. Tokunaga A, Saika M, Senba E. 5-HT_{2A} receptor subtype is involved in the thermal hyperalgesic mechanism of serotonin in the periphery. Pain. June 1998:76(3) 349-355.
H. Yao G L, Tohyama M, Senba E. Histamine-caused itch induces Fos-like immunoreactivity in dorsal horn neurons: effect of morphine pretreatment. Brain Res. 1992 Dec. 25; 599(2):333-7.
I. Kanayama M, Hashimoto T, Shigenobu K, Yamane S. Efficacy of serotonin receptor blocker for symptomatic lumbar disc herniation. Clin Orthop Relat Res. 2003 June;(411):159-65.

The description and drawings of the present invention provided in the paper should be considered as illustrative only of the principles of the invention. The invention may be configured in a variety of ways and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A sampling device comprising:
an outer wall defining an outer enclosure having an internal space;
an inner wall defining an inner enclosure having an internal space, said inner wall positioned within the internal space of the outer enclosure;
a barrier extending within the internal space of the inner enclosure, the barrier defining a first lumen and a second lumen; and,
a third lumen adjacent the first lumen and the second lumen, said third lumen positioned within the internal space of said outer wall.

2. The sampling device of claim 1, wherein the first lumen comprises an inflow lumen, the second lumen comprises an outflow lumen, and the third lumen comprises an injector lumen.

3. The sampling device of claim 1, wherein the first inner wall has a distal end, and further comprising a membrane coupled to the distal end of the first inner wall.

4. The sampling device of claim 3, wherein said barrier extends substantially parallel to the inner wall.

5. The sampling device of claim 4, wherein said barrier has a distal end, and further comprising a gap between the distal end of said barrier and the distal end of said first inner wall.

6. A sampling device comprising:
a first tube having an outer wall defining an internal space;
a second tube having an inner wall defining an internal space, the second tube located within the internal space of the first tube; and,
a barrier located within the internal space of the second tube, the barrier defining a first lumen and a second lumen.

7. The sampling device of claim 6, further comprising a third lumen adjacent the first lumen and the second lumen.

8. The sampling device of claim 7, said third lumen located within the internal space of said first tube.

9. The sampling device of claim 6, wherein said sampling device is configured to detect a neurological response of an intervertebral disc.

10. The sampling device of claim 6, further comprising a membrane positioned over a distal end of said second tube.

11. The sampling device of claim 10, wherein said first lumen comprises an inflow lumen configured to carry a dialysate mixture or solute to said membrane, said membrane is configured to derive diffusion of a biological agent from a target tissue, and said second lumen comprises an outflow lumen configured to carry the diffused dialysate mixture or solute.

12. The sampling device of claim 11, wherein said target tissue comprises an intervertebral disc.

13. The sampling device of claim 6, wherein said second tube can move longitudinally with respect to said first tube, such that said second tube can extend outside said first tube.

14. A method for sampling an intervertebral disc of a patient utilizing a sampling device, the method comprising:
providing a dialysate mixture or solute in the sampling device; and,
allowing a biological substance of the intervertebral disc to enter the sampling device and diffuse with the dialysate mixture or solute.

15. The method of claim 14, further comprising introducing through the sampling device, a medication or biologic agent to the intervertebral disc, the intervertebral disc providing biological substance that reacts thereto; and allowing the reacting biological substance to enter the sampling device and diffuse with the dialysate mixture or solute.
